(19) United States
(12) Patent Application Publication
(10) Pub. No.: US 2024/0238194 A1
(43) Pub. Date: Jul. 18, 2024

(54) ORODISPERSIBLE EFFERVESCENT TABLET COMPRISING BUDESONIDE FOR USE IN THE TREATMENT OF EOSINOPHILIC ESOPHAGITIS

(71) Applicant: Dr. Falk Pharma GmbH, Freiburg (DE)

(72) Inventors: Rudolf WILHELM, Bischweier (DE); Ralph MUELLER, Freiburg (DE); Roland GREINWALD, Kenzingen (DE); Markus PROELS, Freiburg/Breisgau (DE)

(73) Assignee: Dr. Falk Pharma GmbH, Freiburg (DE)

(21) Appl. No.: 18/562,212

(22) PCT Filed: May 19, 2022
(86) PCT No.: PCT/EP2022/063514

§ 371 Date: Nov. 17, 2023

(30) Foreign Application Priority Data
May 20, 2021 (EP) 21174941.1

(51) Int. Cl. A61K 9/46; A61K 9/00; A61K 9/20; A61K 31/58; A61P 1/04

(52) U.S. Cl. CPC A61K 9/0007; A61K 9/0056; A61K 9/2009; A61K 9/2013; A61K 31/58; A61P 1/04

(57) ABSTRACT

Orodispersible effervescent tablets comprising budesonide are administered to patients suffering from eosinophilic esophagitis whereby a complete mucosal healing (i.e., deep endoscopic AND deep histological remission) and even a deep disease remission (i.e., deep clinical AND deep endoscopic AND deep histological remission) is achieved by a treatment regimen of administering said orodispersible effervescent tablets for at least six weeks to twelve weeks for the induction period and thereafter up to three a years as maintenance treatment.

### PRIORITY

This application corresponds to the U.S. National phase of International Application No. PCT/EP2022/063514, filed May 19, 2022, which, in turn, claims priority to European Patent Application No. 21174941.1 filed May 20, 2021, the contents of which are incorporated by reference herein in their entirety.

### BACKGROUND OF THE PRESENT INVENTION

Eosinophilic esophagitis (EoE) is conceptually defined in the revised American Gastroenterological Association's consensus recommendations for diagnosis and treatment of EoE as a “chronic, antigen-/immune-mediated esophageal disease characterized by an eosinophil-predominant inflammation of the esophageal mucosa causing symptoms of esophageal dysfunction”. Currently, EoE is defined by the combination of clinical symptoms of esophageal dysfunction, most commonly dysphagia and chest pain, and the histological finding of at least 15 eosinophils (eos) in one high power microscopic field (hpf) from a biopsy from the esophageal mucosa and should be diagnosed by clinicians, taking into consideration all clinical and pathologic information; neither of these parameters should be interpreted in isolation.

Eosinophilic esophagitis is mainly found in industrialized countries such as Europe, the United States, Canada, and Australia, with an increasing prevalence and incidence. In a recent systemic review by Navarro P, Arias A, et al. Aliment Pharmacol Ther 2019; 49:1116-25, the pooled prevalence of EoE was 34.4 cases per 100 000 inhabitants (95% CI, 23.1-47.5), with pooled EoE incidence rates of 6.6/100 000 person-years (95% CI, 3-11.7) in children and 7.7/100 000 (95% CI, 1.8-17.8) in adults. Males are more commonly affected than females, accounting for about 76% of adult patients and 66% of pediatric patients. Eosinophilic esophagitis affects all age groups with a peak between the age of 20 and 50.

In adults, the most common presenting clinical symptoms are intermittent dysphagia for solids (range: 29%-100% depending on the series) and food impaction (range: 25%-100%). One report found that EoE was responsible for 50% of cases of esophageal food impaction in one institution. Gastro-Esophageal-Reflux-Disease (GERD)-like symptoms are also reported (range: 7%-100%). Chest pain that is unrelated to swallowing activity (range: 1%-58%), abdominal pain (range: 3%-25%); diarrhea and weight loss are reported in some patients. Many adults had long-standing symptoms including recurrent food impactions prior to the diagnosis of EoE.

Eosinophilic esophagitis tends to be a chronic disease with persistent or relapsing symptoms, but is not characterized by mucosal ulceration or destruction and does not appear to limit life expectancy. To date, esophageal strictures and small caliber esophagus, often resulting in trouble of swallowing (i.e., dysphagia) up to food impaction, have been the major complications identified. When these findings are encountered, either radiologically or at endoscopy, a high index of suspicion should be raised for EoE, and mucosal biopsy specimens should be obtained. Careful long-term follow-up is advised. Other chronic problems include failure to thrive and feeding intolerance in children. However, if left for longer time untreated, strictures develop by esophageal remodeling.

Dellon et al., Gastroenterology, 2012, 143, pp 321-324 performed a randomized trial to compare nebulized and then swallowed (NEB) versus viscous topical corticosteroid (OVB) treatments for eosinophilic esophagitis (EoE). The patients received budesonide 1 mg twice daily either nebulized and then swallowed (NEB) or as an oral viscous slurry (OVB) for eight weeks. After treatment, eosinophilic counts markedly improved in the oral viscous slurry treated group but not in the nebulized and then swallowed group. The study illustrates the importance of adequate esophageal targeting for efficacy.

A highly favorable effect of systemic and topical corticosteroids has been demonstrated for the treatment of inflammatory diseases. However, treatment with corticosteroids is associated with side effects. Long-term treatment with systemic corticosteroids is burdened with severe adverse effects, mainly depending on the duration and the dosage of the medication.

In contrast, budesonide is acting dominantly locally due to its affinity to the tissue and its high grade first pass liver metabolism, and showed a positive benefit/risk ratio in EoE. However, it is unclear, whether long-term use is able to maintain clinico-pathological remission.

As already shown by Dellon et al., Gastroenterology, 2012, the mucosal medication contact time, measured by scintigraphy, was higher for an oral viscous budesonide suspension group (OVB) than the nebulized and then swallowed budesonide (NEB) group (P<0.005). OVB provided a significantly higher level of esophageal exposure to the therapeutic agent, which correlated with lower eosinophil counts. With this oral viscous slurry formulation and a budesonide 1 mg twice daily application, 64% of patients versus 27% under NEB achieved complete histological remission (i.e., <1 eosinophil/high power field), mainly due to prolonged contact time.

Greuter et al. (American Journal of Gastroenterology, October 2017, Vol. 112, Issue 10, pp 1527-1535) describe a long-term treatment of eosinophilic esophagitis with swallowed topical corticosteroids. In this study corticosteroids prepared for the treatment of asthma were used off-label as viscous corticosteroid solutions. Viscous suspensions containing 1.0 mg fluticasone or budesonide were administered twice daily. It has been reported that a “deep remission” (combination of clinical, endoscopic and histological remission), was achieved after a median of 89 weeks of treatment. In detail, “deep remission” was defined as “Long-term clinical remission: lack of any EoE-attributed symptoms (dysphagia and non-swallowing-associated retrosternal pain) under unrestricted nutritional habits for at least 6 months; Endoscopic inflammatory remission: complete absence of inflammatory signs, in particular white exudates, furrows, and edema; Histological inflammatory remission: peak eosinophil count<5 eosinophils/hpf”. However, this definition still allowed some inflammation (i.e. 1-4 eos/hpf) and some endoscopic signs of stricturing disease and is therefore less stringent than the definitions for remission used for the present invention. Examples as presented below clearly demonstrate the superior efficacy of the present intervention compared to the published state of the art.

Orodispersible tablets such as described in EP 2 886 108 and EP 3 086 782 are pharmaceutically well-accepted formulations suitable for esophageal targeting which allows small amounts of budesonide following the disintegration of the orodispersible tablet in the mouth during a period of about 2 minutes up to 20 minutes, preferably about 2-5 minutes to reach the esophagus. The embodiments described in the two European patents are preferably used according to the present invention.

### SUMMARY OF THE PRESENT INVENTION

In the present invention the budesonide is preferably administered as an orodispersible effervescent tablet which contains 0.5 mg or 1.0 mg or 2.0 mg budesonide. The effervescent action is caused by the salt of at least one pharmaceutically acceptable acid which in an aqueous environment, in particular in the mouth containing saliva, can release a gas which is preferably CO₂ in cooperation with a further acid. The effervescent tablet contains the salt of a further weak acid or a further weak acid that decreases the pH value in the aqueous solution. The preferably used orodispersible effervescent tablet has a mass between 100 mg to 200 mg, and preferably between 130 mg to 150 mg. In a preferred embodiment the orodispersible effervescent tablet contains sucralose instead of aspartame in an amount of about 0.1 to 1.0% by weight based on the weight of the orodispersible effervescent tablet. The salt of the pharmaceutically acceptable acid can release a gas in the aqueous (saliva) environment of the mouth together with an acid. An essential component of the orodispersible effervescent tablet is at least one of the carbonates selected from the group consisting of NaHCO₃, Na₂CO₃, KHCO₃, K₂CO₂ and CaCO₃ or mixtures thereof. The salt of the further pharmaceutically acceptable weak acid that decreases the pH value in the aqueous (saliva) solution is disodium citrate, monosodium citrate or a mixture thereof.

The orodispersible effervescent tablet which is preferably used according to the teaching of the present invention is laid on the tip of the tongue and disintegrates slowly driven by the reaction between the saliva and the effervescent system. The tablet is comparatively small and has a diameter of about 5-10 mm and a height of 1.5-3.0 mm. The tablets are prepared by applying a suitable pressure in the tableting process such that the fracture strength is between about 10 N to 100 N and the friability is at most 5%.

### BRIEF DESCRIPTION OF THE FIGURES

FIG. 1 presents a graphic summary of the results observed in the EOS-1 clinical trial described further in the examples, wherein treatment with orodispersible effervescent tablets comprising 1 mg budesonide administered twice a day (BID) was compared against placebo.

FIG. 2 graphically presents the percentage of radioactivity observed over time in the esophagus with an oral viscous suspension (top line) or an orodispersible effervescent tablet (inset graph).

FIG. 3 graphically presents the change from baseline to week 6 (LOCF) in the esophageal distensibility upon treatment with orodispersible effervescent tablets comprising 1 mg budesonide administered twice a day (BID) or placebo.

FIG. 4 graphically presents the change from baseline (maintenance) to week 48 (LOCF) in the esophageal distensibility upon treatment with orodispersible effervescent tablets comprising 1 mg budesonide BID, 0.5 mg budesonide BID, or placebo.

FIG. 5 graphically presents the change from screening (pre-treatment) to end of treatment in the esophageal distensibility during a 3-years maintenance treatment with budesonide orodispersible tablets.

### DETAILED DESCRIPTION OF THE PREFERRED EMBODIMENTS

Eosinophilic esophagitis (EoE) is a chronic, inflammatory disease of the esophagus. For diagnosis of EoE the following criteria are considered:
presence of symptoms of esophageal dysfunction and predominant eosinophilic inflammation of the esophagus;
endoscopic evaluations, in particular regarding EoE associated endoscopic signs like edema, rings, exudates, longitudinal furrows and/or strictures.

The eosinophil is a late-phase inflammatory cell that plays an important role in tissue repair and remodeling. Eosinophilic inflammation may induce fibrosis with the consequence of luminal narrowing and stricture formation finally resulting in an impairment of esophageal functions. It is assumed that EoE is a progressive fibrostenotic disease since there are indications that the extent of fibrosis has been positively associated with disease duration. As the disease progresses, a mixture of inflammatory and fibrotic features can be observed.

One potential diagnostic tool for assessing disease activity and disease progression that provides information on esophageal remodeling is endoscopic imaging. Characteristic esophageal abnormalities for EoE which can be evidently seen during endoscopy include:
edema (loss of vascular markings)
rings (trachealization)
exudates (white plaques)
longitudinal furrows (vertical lines) and
strictures.

These features can be assessed and graded in their presence and intensity by using the validated endoscopic reference score (called EREFS) as being published by Hirano et al. Gut. 2013; 62(4):489-95.

### Major Features:

Fixed rings (also referred to as concentric rings, corrugated esophagus, corrugated rings, ringed esophagus, trachealisation)
Grade 0: none
Grade 1: mild (subtle circumferential ridges)
Grade 2: moderate (distinct rings that do not impair passage of a standard diagnostic adult endoscope (outer diameter 8-9.5 mm)
Grade 3: severe (distinct rings that do not permit passage of a diagnostic endoscope)

Exudates (also referred to as white spots, plaques)
Grade 0: none
Grade 1: mild (lesions involving≤10% of the esophageal surface area)
Grade 2: severe (lesions involving>10% of the esophageal surface area

Furrows (also referred to as vertical lines, longitudinal furrows)
Grade 0: absent
Grade 1: present

Edema (also referred to as decreased vascular markings, mucosal pallor)
Grade 0: absent (distinct vascularity present)
Grade 1: loss of clarity or absence of vascular markings

Stricture
Grade 0: absent
Grade 1: present

### Minor Feature:

Crêpe paper esophagus (mucosal fragility or laceration upon passage of diagnostic endoscope but not after esophageal dilation).
Grade 0: absent
Grade 1: present

The total score ranges from 0-9 points.

A total EREFS score of ‘0 points’ defines ‘deep endoscopic remission’, i.e. the complete absence of any inflammatory or fibrostenotic signs of EoE. The endoscopic assessment of the esophageal features of eosinophilic esophagitis is measured according to the teaching of Hirano et al. A deep endoscopic remission according to the present invention is obtained when a total EREFS score of 0 points is achieved. Therefore, a high standard is applied to the definition of deep endoscopic remission as all the features lined out above must be graded as 0. This definition of “deep endoscopic remission” is much more stringent and harder to achieve as the one published by Greuter et al. (American Journal of Gastroenterology, October 2017, Vol. 112, Issue 10, pp 1527-1535) described above.

Since the endoscopic determination allows only a visual inspection of mucosal inflammatory and fibrotic signs, but is not suitable to detect changes in organ function of the esophagus itself, an objective and measurable system can be used complementary to endoscopy to study changes in esophagus function. The Endolumenal Functional Lumen Imaging Probe (EndoFLIP®, Crospon Ltd., Galway, Ireland) is a commercial lumen imaging catheter, which is emerging as a new standard for assessment of luminal dimensions and compliance. The balloon catheter is deployed alongside an endoscope during patient evaluation, and inflated with fluid to a low distending pressure of 40 mmHg or up to 60 mL. The device contains a cylindrical bag that can be inflated with a conductive fluid while simultaneously CSA (cross-sectional area) and intra-bag pressure and applied volume are measured to calculate the distensibility of the esophagus which mimics its function.

It is assumed that the distensibility evaluation of the esophagus using the functional lumen imaging probe (FLIP) offers an objective measure to characterize fibrostenotic remodeling processes in patients with EoE and the effect on these fibrostenotic remodeling processes under treatment with the present invention. Left untreated, the chronic inflammation in EoE is believed to progress to fibrostenotic changes manifested as the characteristic ringed and strictured esophagus that can result in esophageal mechanical obstruction and associated symptoms, in particular dysphagia.

The therapeutic success of a treatment can be determined by achievement of deep endoscopic remission defined as a modified EREFS score of ‘0 points’ and by its physiological consequence, i.e. an increased esophageal distensibility as measured by the EndoFLIP® technique. The functional lumen-imaging probe (FLIP) utilizes high-resolution impedance planimetry to render a 3-dimensional approximation of intraluminal esophageal anatomy during volumetric distension. Frequently, EoE patients have reduced esophageal distensibility compared to a healthy population.

The present invention provides a treatment regimen for an orodispersible effervescent tablet comprising budesonide as described herein for use in the treatment of eosinophilic esophagitis wherein a deep endoscopic remission (EREFS=0 points) and deep histological remission (eos=0/hpf) is achieved by the twice daily administration of the orodispersible effervescent tablet for a duration of at least six weeks (42 days) and also maintained over a period of at least 48 weeks. Deep endoscopic remission and deep histological remission is designated in the sense of the present invention as “complete mucosal healing”.

Adding to this “complete mucosal healing” definition the complete absence of the leading EoE clinical symptoms (i.e., problems to swallow (dysphagia) and pain during swallowing “odynophagia”), both rated as “0”, each on 0-10 points Numerical Rating Scales (NRS) [with 0 point indicating “no symptoms at all” and 10 points indicating “the most severe symptom”) on each day in the last 7 days) leads to the most stringent endpoint definition for achieving at the same time deep clinical, deep endoscopic and deep histological remission which is designated in the sense of the present invention as “Deep disease remission”).

According to a preferred embodiment of the present invention the treatment regimen comprises an induction period and a maintenance period. Surprisingly, during the induction period of a duration of 6-12 weeks complete mucosal healing is already achieved. During the induction period the patient is treated with orodispersible effervescent tablets as described herein whereby twice daily, preferably in the morning and in the evening a tablet comprising 1 mg budesonide is administered. In an alternative embodiment the induction treatment can be obtained with a once daily administration of an orodispersible effervescent tablet containing 2 mg budesonide.

After the induction period a maintenance period follows which lasts from 48 weeks up to 3 years. During the maintenance period the orodispersible effervescent tablet is preferably administered twice daily with a 1 mg budesonide tablet. In another embodiment an orodispersible tablet containing 0.5 mg budesonide is administered twice daily. In an alternative embodiment the maintenance treatment can be obtained with a once daily administration of an orodispersible effervescent tablet containing either 1 mg or 2 mg budesonide.

One of the most remarkable effects obtainable with the present treatment regimen is that fibrostenotic changes of the esophagus can be avoided and in part reverted. This is an important aspect since as soon as the esophagus has started the process of tissue conversion, treatment of the patient may become very difficult and long-lasting symptoms of esophageal dysfunction including frequent bolus impaction might occur.

Another surprising effect is that complete mucosal healing and even deep disease remission can be obtained with the present invention after a short induction period and be maintained over a long time.

The dosage regimen of the present invention comprises the administration of an orodispersible effervescent tablet to the patient in doses of 0.5 mg or 1.0 mg budesonide twice a day or 2 mg budesonide once daily. The application is preferably applied in a dosing regimen wherein 1 mg budesonide is applied as one orodispersible tablet comprising 1 mg budesonide twice daily. Alternatively, two tablets comprising 0.5 mg budesonide in the morning and one tablet comprising 1 mg budesonide or two tablets comprising 0.5 mg budesonide in the evening can be administered. The induction treatment should be performed over a period of at least six weeks (42 days) up to 12 weeks (84 days) for induction of deep endoscopic remission and in addition complete mucosal healing. The induction treatment is followed by a maintenance treatment with a duration of 48 weeks up to 3 years as decided by the treating physician.

The disintegration of each orodispersible effervescent tablet in the mouth occurs in a time span of at least 2 minutes to about 10 minutes, preferably in a time span extending from at least 2 minutes to about 5 minutes, but can take up to 20 minutes. This delayed disintegration of the orodispersible effervescent tablet results in a continuous production of saliva in which budesonide is suspended during disintegration. During this prolonged disintegration time, the patient swallows on average 10-times the budesonide containing saliva. It is assumed that the naturally occurring mucoadhesive agents in the saliva enable a stable coating of the esophagus whereby the budesonide is brought into close contact to the cells of the outer surface of the esophagus allowing a fast uptake of budesonide.

Up to now, it was assumed that stopping and reverting the fibrostenotic remodelling is a lengthy process, which needs time. Therefore, it was unclear whether drug treatment that is able to induce and, in addition, to maintain deep endoscopic remission over a longer period can also stop and partially revert fibrostenotic remodelling. However, achieving deep endoscopic remission increases the likelihood of stopping the fibrostenotic remodeling process and even revert it. Thus, an improvement of esophageal distensibility towards normal values might be seen.

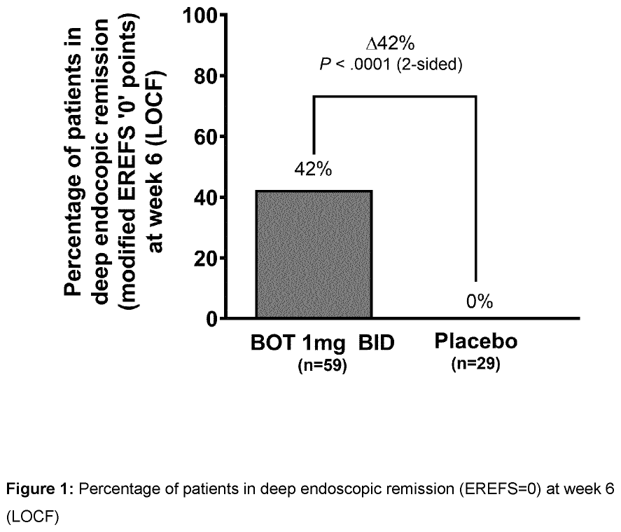

Surprisingly, the treatment regimen, namely an at least 6-week treatment with a budesonide 1 mg orodispersible effervescent tablet administered twice daily to EoE patients with active disease was also able to bring significantly more patients into deep endoscopic remission compared to placebo, as shown in FIG. 1.

At baseline of the induction of remission treatment, none of the patients was in deep endoscopic remission (EREFS=0 points). Treatment with orodispersible effervescent tablets comprising 1 mg budesonide administered twice a day (BID) had the consequence that 42% of the patients achieved deep endoscopic remission at week 6 (42 days after start of the treatment) compared with 0% of patients who received placebo.

This unexpected rapid success (i.e., achievement of deep endoscopic remission) of targeted treatment with orodispersible effervescent tablets is also mirrored by the observed quick and high rates (89.8%) of deep histological remission (i.e., ‘0’ eos/hpf in all biopsies) achieved by a 6-week treatment with budesonide 1 mg orodispersible effervescent tablet twice daily compared to 0% under placebo.

This rapid onset and the extraordinary efficacy might be well explained by the special pharmaceutical formulation, its release and esophageal targeting profile of the present invention.

In a recent scintigraphic study it could surprisingly be shown that an ¹¹¹Indium-labelled orodispersible effervescent tablet comprising 1 mg budesonide provides a substantially longer retention of the radioactivity (and thus also of the budesonide) at the esophageal mucosa than 5 ml of an ¹¹¹Indium-labelled oral viscous budesonide suspension [0.2 mg/ml].

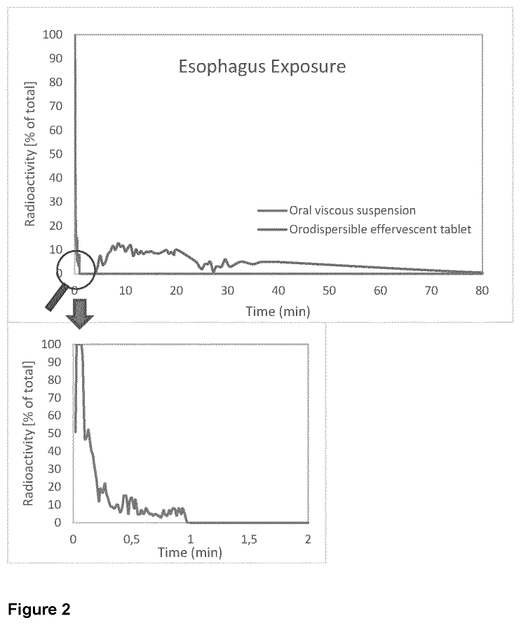

A representative picture of the percentage of radioactivity observed over time in the esophagus is shown in FIG. 2. The study was performed with healthy volunteers (N=10). Each volunteer obtained each of the two preparations whereby the distribution of the indium was observed with a suitable scintigraphic camera. As regions of particular interest, the oral/buccal area, the esophagus and the stomach were defined. At different periods of time, the radioactivity in the esophagus was set in relation to the total radioactivity.

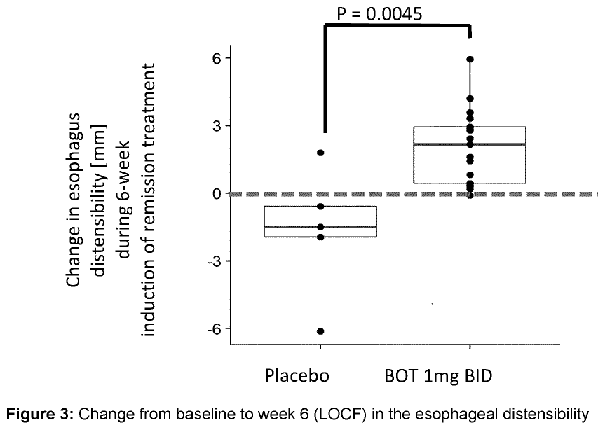

In addition, and also not expected due to the short treatment period of only 6 weeks, the esophageal distensibility (i.e., the diameter of the esophagus) also significantly improved compared to placebo as depicted in FIG. 3.

FIG. 3 shows the change from baseline in the distensibility of the esophagus [measured in mm]. The values were measured 42 days after start of the treatment by the EndoFLIP test system. Under placebo treatment, the distensibility of the esophagus decreased (indicating a narrowing of the esophageal diameter), whereas the distensibility in the group receiving the orodispersible effervescent tablet comprising 1 mg budesonide administered twice daily was significantly improved (indicating a widening of the esophageal diameter).

This significant improvement in the esophagus diameter within such a short time of only 6-weeks could only be explained by the extremely high local anti-inflammatory activity of the budesonide orodispersible effervescent tablet. This is achieved by the mode of release and delivery of budesonide from the orodispersible effervescent tablet to the esophageal mucosa and its prolonged contact time to the mucosa.

In addition, and unexpected due to the short treatment period of only 6 weeks, the new treatment regimen of budesonide 1 mg orodispersible effervescent tablet formulation when administered twice daily led to deep histological remission rates of 90%. Again, this is caused by the mode of release and delivery of budesonide from the orodispersible effervescent tablet to the esophageal mucosa and its prolonged contact time to the mucosa.

Beside the unexpected high efficacy in improvement of the esophageal distensibility as well as in the induction of deep endoscopic remission and deep histological remission, both achieved already after only a 6-week treatment course with budesonide 1 mg orodispersible effervescent tablets (twice daily), recent long-term data showed that budesonide 1 mg or 0.5 mg orodispersible effervescent tablets (twice daily) were able to maintain the therapeutic success also over a period of 48 weeks in a placebo-controlled double-blind trial, as presented in Table 1 below:

TABLE 1
Course of “deep endoscopic remission” over
48-weeks maintenance treatment (FAS-DB)

| | Proportion of patients with deep endoscopic | | |
|---|---|---|---|
| | remission during a 48-weeks maintenance treatment | | |
| | BOT 0.5 mg BID | BOT 1.0 mg BID | Placebo |
| | n = 68 | n = 68 | n = 68 |
| Week 0 | 34 (50.0%) | 34 (50.0%) | 35 (51.5%) |
| Week 48 | 36 (52.9%) | 39 (57.4%) | 4 (5.9%) |

*BID ≙ twice daily

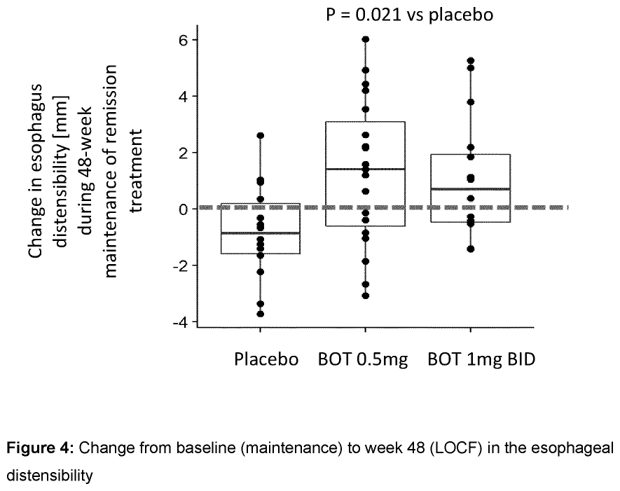

In addition, the esophageal distensibility (i.e., the diameter of the esophagus) also significantly further improved under treatment with budesonide orodispersible effervescent tablets over 1-year maintenance treatment compared to placebo (p=0.021) as shown in FIG. 4. Although the distensibility of the esophagus further improved also with the lower dosage of 0.5 mg budesonide it should be mentioned that the statistic variance of the values obtained with 0.5 mg budesonide is substantially higher at a treatment of 0.5 mg budesonide twice daily. Therefore, it is preferred to administer orodispersible effervescent tablets (BUL) containing 1 mg budesonide twice daily (BID) also during maintenance therapy.

Surprisingly, during a prolonged 2-years open-label extension maintenance treatment with budesonide orodispersible effervescent tablets, only 2 of 166 patients (1.2%) experienced a clinical relapse, or a food impaction which needed endoscopic intervention or needed an endoscopic dilation. This unexpected change in the natural course of the disease was also reflected by the fact that the rate of patients in deep endoscopic remission at the beginning of this 2-year open-label treatment phase (55 out of 82 patients (67.1%) who were endoscoped) did not worsen during this 2-year treatment phase. Even more, it was a surprise that by a total treatment of 3 years with budesonide orodispersible effervescent tablets it was possible to maintain deep histological remission, i.e. ‘0’ eos/mm² hpf in about 40 of 49 patients (81.6%) and even to maintain complete mucosal healing in about 39 of 56 patients (69.6%).

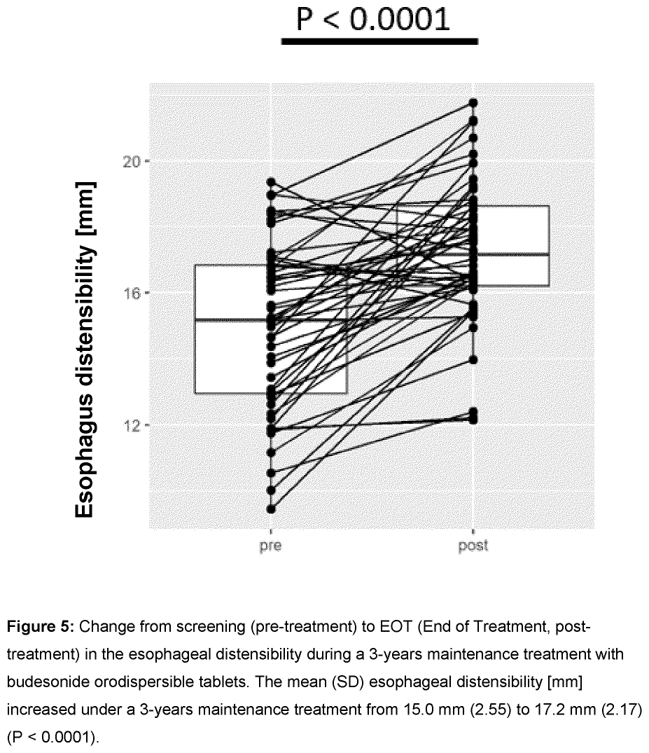

Moreover, a 3-years continuous treatment with BUL led to a sustained and clinically relevant improvement in the esophageal distensibility as being shown in FIG. 5, and which indicates that orodispersible effervescent tablets (BUL) containing 1 mg or 0.5 mg budesonide twice daily (BID) are able to change the natural course of EoE, and can stop and revert the fibrostenotic remodeling and successfully prevent the re-occurrence of such events.

In addition, with a 6-week induction of remission treatment followed by a prolonged, up to 3-years maintenance treatment with budesonide orodispersible effervescent tablets, it was possible to achieve and continuously maintain treatment success even for the totality of three different deep remission criteria (modified and adapted to Greuter et al. (American Journal of Gastroenterology, October 2017, Vol. 112, Issue 10, pp 1527-1535) in the vast majority of patients (see Table 2).

TABLE 2
Maintaining “deep” remission criteria
over 3-years of maintenance treatment

| | Total |
|---|---|
| Rate of patients maintained in deep endoscopic remission | 32 (91.4%) |
| from DB Baseline to OLE2 EOT^{§} | n = 35 |
| Rate of patients maintained in deep clinical remission | 22 (88.0%) |
| from DB Baseline to OLE2 EOT^{$} | n = 25 |
| Rate of patients maintained in deep remission, i.e., deep | 8 (66.7%)-- |
| clinical, deep endoscopic, and histological remission from | n = 12 |
| DB Baseline to OLE2 EOT^{#&} | |

Deep endoscopic remission was defined as: total modified EEsAI endoscopic instrument subscores fulfill the following criteria: fixed rings = ‘Grade 0: none’ or ‘Grade 1: mild’, exudates = ‘Grade 0: none’, furrows = ‘Grade 0: absent’, and edema = ‘Grade 0: absent’.
Deep clinical remission was defined as NRS for dysphagia is ‘0’ and the NRS for pain during swallowing is ‘0’
Deep remission was defined as when the patient fulfills the following three criteria: Deep clinical, deep endoscopic, and histological remission (based on the peak number of <5 eos per hpf).

Using the most stringent “deep disease remission” definition [i.e., deep clinical remission: dysphagia and odynophagia both rated as “0”, each on 0-10 points Numerical Rating Scales (with 0 point indicating “no symptoms at all” and 10 points indicating the most severe symptom) each day in the last 7 days; deep endoscopic (i.e. EREFS=0 points) and deep histological remission (i.e., eos=0/hpf)], and an up to 3-years maintenance treatment with budesonide orodispersible effervescent tablets, it was possible to achieve and continuously maintain such very stringent defined “deep disease remission” in 14 of 20 patients (70%).

FIG. 5 shows the change from screening to EOT (End of Treatment) in the esophageal distensibility during a 3-years maintenance treatment with budesonide orodispersible tablets. The single data points reflect different patients. From the statistical analysis it turned out that the mean (SD) esophageal distensibility, measured in mm increased under a 3-years maintenance treatment, whereby the distensibility increased from an average value of 15.0 mm±(2.55) obtained at screening, i.e. before treatment, to 17.2 mm±(2.17 (P<0.0001). FIG. 5 provides therefore evidence that on the long term, the esophageal distensibility improved substantially by the treatment according to the invention.

### EXAMPLES

The results as shown in FIGS. 1-5 were obtained by clinical trials which were performed as follows:

### Example 1

### EOS-1 Induction of Remission

BACKGROUND & AIMS: This trial was a randomized, placebo-controlled trial to assess the effectiveness and tolerability of an orodispersible effervescent tablet comprising budesonide (BOT; now called ‘budesonide orodispersible tablet’) formulation, i.e., the present invention, which allows the drug to be delivered to the esophagus in adults with active EoE.

METHODS: EOS-1 was a pivotal, randomized, double-blind induction of remission trial in 88 adult EoE patients performed in several European countries. Patients were randomly assigned to groups that received BOT (1 mg twice daily; n=59) or placebo (n=29) for 6 weeks. The primary end point was complete remission, based on clinical and histologic factors, including dysphagia and odynophagia severity≤2 points on a scale of 0-10 (‘0’=no symptoms; ‘10’=most severe symptoms) on each of the 7 days before the end of the double-blind phase and a peak eosinophil count<5 eosinophils/high power field. Patients who did not achieve complete remission at the end of the 6-week double-blind phase were offered 6 weeks of open-label treatment with BOT (1 mg twice daily).

RESULTS: At 6 weeks, 58% of patients given BOT were in complete clinical and histological remission compared with no patients given placebo (P<0.0001). The secondary end point of histologic remission was achieved by 93% of patients given BOT vs no patients given placebo (P<0.0001). After 12 weeks, 85% of patients had achieved remission. Six-week and 12-week BOT administration were safe and well tolerated; 5% of patients who received BOT developed symptomatic, mild candida, which was easily treated with an oral antifungal agent.

CONCLUSIONS: In a randomized trial of adults with active EoE, we found that orodispersible effervescent tablets comprising budesonide were significantly more effective than placebo in inducing clinical and histologic remission.

### Example 2

### EOS-2 Maintenance of Remission Trial

BACKGROUND & AIMS: Eosinophilic esophagitis (EoE) is a chronic inflammatory disorder. Swallowed topical-acting corticosteroids are effective in bringing active EoE into remission. However, it is not clear whether these drugs are effective for long-term maintenance of remission.

METHODS: EOS-2 was a double-blind trial to compare the efficacy and safety of 2 dosages of an orodispersible effervescent tablet comprising budesonide (BOT; now called ‘budesonide orodispersible tablet’) vs placebo in maintaining remission of EoE over 48 weeks, followed by a 2-years open-label extension phase for continous maintenance therapy with recommended BOT 0.5 mg BID (if needed dose could be increased to BOT 1 mg BID). Maintenance of remission was defined as absence of clinical and histologic relapse and no premature withdrawal for any reason. Two hundred and four adults with EoE in clinical and histologic remission, from 29 European study sites, were randomly assigned to groups given BOT 0.5 mg twice daily (n=68), BOT 1.0 mg twice daily (n=68), or placebo twice daily (n=68) for up to 48 weeks.

RESULTS: At end of double-blind 48-weeks treatment, 73.5% of patients receiving BOT 0.5 mg twice daily and 75% receiving BOT 1.0 mg twice daily were in persistent remission compared to 4.4% of patients in the placebo group (P<0.001 for both comparisons of BOT with placebo). Median time to relapse in the placebo group was 87 days. The frequency of adverse events was similar in the BOT and placebo groups. Morning serum levels of cortisol were in the normal range at baseline and did not significantly change during treatment. Four patients receiving BOT developed asymptomatic, low serum levels of cortisol. Clinically manifested candidiasis was suspected in 16.2% of patients in the BOT 0.5 mg group and in 11.8% of patients in the BOT 1.0 mg group; all infections resolved with treatment.

CONCLUSIONS: In a phase 3 trial, up to 48 weeks of treatment with BOT (0.5 mg or 1.0 mg twice daily) was superior to placebo in maintaining remission of EoE. Both dosages were equally effective and well tolerated.

1-18. (canceled)

19. A method of treating eosinophilic esophagitis in a patient in need thereof, said method comprising the step of daily administering to said patient at least one orodispersible effervescent tablet comprising budesonide at least until complete mucosal healing is achieved.

20. The method of claim 19, wherein said orodispersible effervescent tablet comprises 0.5 mg budesonide and is administered twice daily.

21. The method of claim 19, wherein said orodispersible effervescent tablet comprises 1 mg budesonide and is administered twice daily.

22. The method of claim 21, wherein said twice daily administration comprises a first orodispersible effervescent tablet comprising 1.0 mg budesonide administered in the morning and a second orodispersible effervescent tablet comprising 1.0 mg budesonide administered in the evening.

23. The method of claim 19, wherein said orodispersible effervescent tablet comprises 2 mg budesonide and is administered once daily.

24. The method of claim 19, wherein said complete mucosal healing is achieved within 6-12 weeks of treatment.

25. The method of claim 19, wherein said complete mucosal healing is evidenced by increased esophageal distensibility.

26. The method of claim 19, wherein said complete mucosal healing is characterized by deep endoscopic and deep histological remission.

27. The method of claim 26, wherein said deep endoscopic remission defined as 0 points of the endoscopic reference score.

28. The method of claim 26, wherein said deep histological remission defined as ‘0’ eos/hpf in all biopsies.

29. The method of claim 19, wherein said complete mucosal healing is characterized by a deep clinical remission defined as an NRS for dysphagia of ‘0’ and an NRS for pain during swallowing of ‘0’.

30. The method of claim 19, wherein said treatment is composed of (i) a 6-12 week induction period, after which complete musical healing is achieved, and (ii) a maintenance period of up to 144 weeks.

31. The method of claim 30, wherein said maintenance period ranges from span six weeks up to three years.

32. The method of claim 30, wherein, during said induction period, said orodispersible effervescent tablet is administered twice a day, once in the morning and again in the evening.

33. The method of claim 19, wherein said complete mucosal healing is characterized by deep disease remission evidenced by a combination of:
(i) deep clinical remission with dysphagia and odynophagia both rated as “0” on an NRS scale, wherein 0 point indicates “no symptoms at all”, each day in a 7 day period,
(ii) deep endoscopic remission with EREFS=0 points, and
(iii) deep histological remission characterized by eos=0/hpf.

34. The method of claim 33, wherein said deep disease remission is maintained over 3 years.

35. The method of claim 19, characterized in that dissolution of said orodispersible effervescent tablet occurs by the action of saliva in the mouth within a time span of 2 to 20 minutes.

36. The method of claim 19, characterized in that dissolution of said orodispersible effervescent tablet occurs by the action of saliva in the mouth within a time span of 2 to 5 minutes.
